(19) United States
(12) Patent Application Publication
(10) Pub. No.: US 2014/0120597 A1
(43) Pub. Date: May 1, 2014

(54) METHOD TO PRODUCE ETHANOL USING WHOLE STILLAGE

(71) Applicant: Golden Corn Technologies, L.L.C., Oskaloosa, IA (US)

(72) Inventor: ROSS D. DALE, Pella, IA (US)

(73) Assignee: Golden Corn Technologies, L.L.C., Oskaloosa, IA (US)

(21) Appl. No.: 14/065,063

(22) Filed: Oct. 28, 2013

Related U.S. Application Data
(60) Provisional application No. 61/720,171, filed on Oct. 30, 2012.

(51) Int. Cl. C12P 7/10

(52) U.S. Cl. CPC C12P 7/10
USPC 435/165

(57) ABSTRACT

A method of producing ethanol is disclosed. The method includes fermentation of carbohydrates from multiple byproduct feedstocks simultaneously during the same fermentation batch by application of a bacterial microbe which converts said carbohydrates to ethanol. A method of producing ethanol from whole stillage is also disclosed comprising converting multiple carbohydrates from multiple feedstocks simultaneously into ethanol without pre-treatment and without added enzymes. A method of producing ethanol with the application of a microbe from the Order Lactobacillales to a byproduct to produce ethanol is also disclosed.

### CROSS-REFERENCE TO RELATED APPLICATIONS

This application claims the benefit of and priority to U.S. Provisional Patent Application Ser. No. 61/720,171 entitled “METHOD TO PRODUCE ETHANOL USING MULTIPLE BYPRODUCT FEEDSTOCKS,” filed Oct. 30, 2012, the entire contents of which is hereby incorporated by reference herein in its entirety.

### FIELD

The invention(s) described herein relate to a method of producing ethanol, and in particular relate to method(s) to produce ethanol using whole stillage byproduct from corn to ethanol dry mill plants.

### BACKGROUND

As is known, ethanol, a renewable fuel, can be produced from agricultural feedstock. Ethanol can be produced from a variety of feedstocks such as sugar cane, bagasse, miscanthus, sugar beet, sorghum, grain, switch grass, barley, hemp, kenaf, potatoes, sweet potatoes, cassaya, sunflower, fruit, molasses, corn, stover, grain, wheat, straw, cotton, other biomass, as well as many types of cellulose waste and harvestings.

As is known, during ethanol fermentation, glucose and other sugars in the corn or other crops are converted into ethanol and carbon dioxide. Bio-ethanol is usually obtained from the conversion of carbon based feedstock. Large scale production of ethanol generally includes the microbial (yeast) fermentation of sugars, distillation, dehydration, and in some instances denaturing.

Currently, many first generation processes for the production of ethanol from corn use only a small part of the corn plant. For example, the corn kernels are taken from the corn plant and only the starch, representing a portion of the dry kernel mass, is transformed into ethanol. Therefore, second generation process are also under development. Two types of second generation processes are known to be under development. The first type uses enzymes and yeast fermentation to convert the plant cellulose into ethanol, while the second type uses pyrolysis to convert the whole plant to either a liquid bio-oil or a syngas.

Under the typical processes, microbial fermentation will currently only work directly with sugars. Currently, only the sugar (e.g. sugar cane) and starch (e.g. corn) portions can be economically converted. Starch and cellulose are molecules that are strings of glucose molecules. Accordingly, it is also possible to generate ethanol out of cellulosic materials. However, in such cases a pretreatment has been necessary that splits the cellulose into glycose molecules and other sugars which subsequently can be fermented to produce cellulosic ethanol. In other words, for many agricultural feedstocks, prior to fermentation, the crops require a pre-treatment such as saccharification or hydrolysis of carbohydrates (e.g., cellulose and starch) into sugars. Enzymes are also typically used to convert starch into sugar.

Accordingly, a need exists for an ethanol production process for production of ethanol from agricultural feedstock which increases the amount of ethanol produced and does not require pretreatment or the addition of enzymes.

### SUMMARY

A method of producing ethanol is disclosed. The method includes fermentation of carbohydrates from multiple byproduct feedstocks simultaneously during the same fermentation batch by application of a bacterial microbe which converts said carbohydrates to ethanol.

A method of producing ethanol from whole stillage is also disclosed. The method includes converting multiple carbohydrates from multiple feedstocks simultaneously into ethanol without pre-treatment and without added enzymes.

A method of producing ethanol including the application of a microbe from the Order Lactobacillales to a byproduct so as to produce ethanol is further disclosed.

These and other features and advantages of devices, systems, and methods according to this invention are described in, or are apparent from, the following detailed descriptions of various examples of embodiments.

### BRIEF DESCRIPTION OF DRAWINGS

Various examples of embodiments of the systems, devices, and methods according to this invention will be described in detail, with reference to the following figures, wherein:

FIG. 1 is a flow chart illustrating one or more examples of a process of the production of ethanol as described herein.

It should be understood that the drawings are not necessarily to scale. In certain instances, details that are not necessary to the understanding of the invention or render other details difficult to perceive may have been omitted. It should be understood, of course, that the invention is not necessarily limited to the particular embodiments illustrated herein.

### DETAILED DESCRIPTION

A method for producing ethanol from byproducts is disclosed. More specifically, a method to produce ethanol is described herein which uses byproduct feedstocks. In particular, ethanol can be produced with the method by using byproducts from the bioenergy industries. The process generally includes fermentation of byproducts with a bacterial microbe.

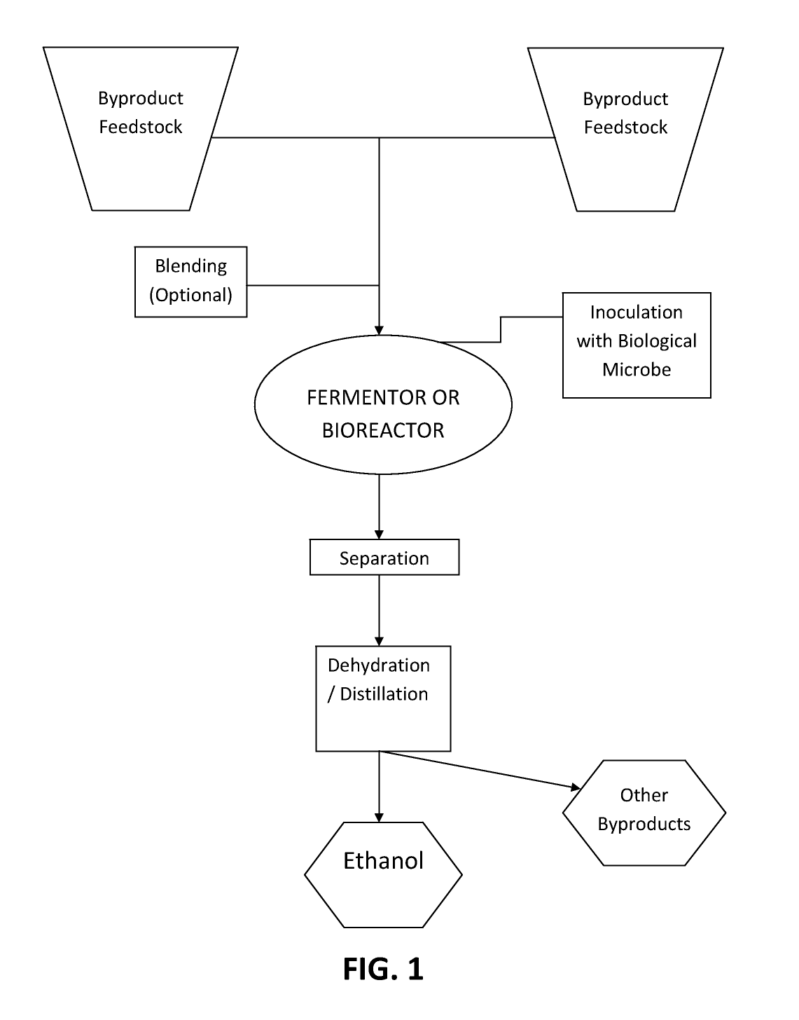

Referring generally to FIG. 1, the method may be practiced using one or more byproduct feedstocks that contain at least one form of fermentable carbohydrate. Preferably, the byproduct feedstock contains multiple carbohydrate types. More preferably, multiple feedstocks are blended to utilize the associated or corresponding carbohydrate for production of ethanol simultaneously on a single fermentation. The byproduct feedstocks may be blended initially before inoculation according to the method. Alternatively, one or more of the byproduct feedstocks can be fed to the fermenter during fermentation to continuously feed the microbes.

A byproduct feedstock is a processing stream from another primary processing method intended to produce a primary chemical. That is, the byproduct feedstock stream is that remaining after separation and purification of the primary chemical from the initial processing stream. For example, whole stillage remains as a byproduct processing stream after ethanol is separated from the mash by distillation. A feedstock as described herein may be any renewable, biological material that can be used directly as a fuel, or converted to another form of fuel or energy product. For example, biomass feedstocks are plant materials used to derive fuels like ethanol, butanol, biodiesel, and other hydrocarbon fuels. Examples of biomass feedstocks include corn starch, sugarcane juice, crop residues, such as corn stover and sugarcane bagasse, purpose-grown grass crops, and woody plants.

Byproducts suitable for use in association with the method include those resultant from any grain processing or animal processing industry and that contain fermentable carbohydrates. Suitable carbohydrates include, for example, cellulose, hemicellulose, residual or resistant starches and glycerol. As indicated, the byproducts in one or more examples of embodiments contain fermentable carbohydrates. The fermentable carbohydrates contained in the byproducts preferably comprise those with carbon chains from 2 to 6 in length and polymers of same. Example fermentable carbohydrates include, but are not limited to, glucose, sucrose, fructose, dextrin, glycerol, acetate, xylose and the like.

Preferably, the byproducts are byproducts resultant from corn wet milling, corn dry grind ethanol production, soybean oil production, any oilseed oil production, starch production from grain, and/or biodiesel production from oilseed oils or animal fats, and combinations of the foregoing. More preferably, the byproducts are chosen from whole stillage resulting from corn dry grind ethanol production, corn wet milling byproducts and/or byproducts from biodiesel production. Even more preferably, the byproducts are from corn ethanol whole stillage. According to one or more examples of embodiments, a byproduct feedstock, such as whole stillage, may be obtained from a corn to ethanol plant. The whole stillage may be obtained, for example, from a commercial corn to ethanol dry grind plant. Whole stillage is residual beer remaining after distillation and removal of ethanol from the beer after primary ethanol fermentation using corn mash.

The byproduct obtained is fermented. The byproduct feedstock may be fermented in a bioreactor. Sanitary conditions are preferred; however, strict sterilization is not required. According to one or more examples of embodiments, the method employs a fermentation with a bacterial microbe.

The organism or bacterial microbe used is able to convert multiple carbohydrate types into ethanol, and accomplishes the conversion during the same fermentation batch. Accordingly, multiple carbohydrates, such as are found in byproducts, are simultaneously converted to ethanol. For example, residual starches, fibers including cellulose and hemicellulose are all converted to ethanol with various extents of conversion. The microorganism is also able to convert multiple carbohydrate types into ethanol without the addition of enzymes. To this end, the microbe is able to convert multiple carbohydrates from multiple feedstocks or raw materials simultaneously and without added nutrients. Moreover, in one or more examples of embodiments, glycerol is completely metabolized by the microbe and may be converted to ethanol. The microbe used is robust and will out-compete contaminating microbes, and as a result production of ethanol using the current method does not require sterilization of the byproduct feedstocks. A bacterial microbe suitable for the purposes provided is preferably from the Order Lactobacillales.

The bacterial microbe of the type set forth herein is introduced into a byproduct or byproduct mixture as described herein and subjected to conditions suitable for fermentation with said microbe in a single fermentation batch. The fermentation process is anaerobic. A description of a suitable example fermentation process, including parameters such as time, temperature, inoculation amount, and so forth, can be found in U.S. Pat. No. 8,173,412, which is incorporated by reference herein in its entirety. To this end, the whole stillage is inoculated with a bacterial microbe of the type described herein and fermented for a period of time. No enzymes are introduced during the processing of the byproduct. Additionally, no pre-treatment occurs on the byproduct, and no nutrients are added.

The fermentation takes place in aqueous solution and the resulting solution after fermentation has a percentage of ethanol content. The ethanol is generally then isolated and purified. According to one example, a combination of adsorption and distillation techniques may be used. Preferably, at the conclusion of the fermentation period, the whole stillage ferment may be separated by common commercial means. For example, separation may occur by use of a decanter, and/or by centrifugation. The separated product may then be subject to dehydration and/or distillation. Like any fermentation reaction, other side products such acetic acid, glycols and other products may be formed which may be removed during the purification or separation of the ethanol.

According to one or more examples of embodiments, fermentation with the microbe and selected byproducts results in ethanol, lactic acid and improved distillers grains feedstuffs. Ethanol accumulation is equal to from about 500 ppm to about 50,000 ppm, wt. basis.

Accordingly, the application of a microbe from the Order Lactobacillales to selected byproducts is capable of producing industrial quantities of ethanol. The use and application of the microbe described herein to produce ethanol from multiple carbohydrate containing feedstocks results in consolidated bio-processing. The application of the process produces renewable fuel without the aid of additional enzymes or pre-treatment of byproducts containing multiple carbohydrates. Moreover, the resultant byproduct from the current method is higher in protein and digestible protein as compared with the initial byproduct feedstock, particularly when the original feedstock is corn ethanol whole stillage. In addition, the byproduct from the current process is higher in rumen undegradable protein, thereby delivering a higher amount of metabolizable protein to the animal when fed to ruminants.

### EXAMPLES

The following Examples are an illustration of one or more examples of embodiments of carrying out the invention and are not intended as to limit the scope of the invention.

### Example 1

Whole stillage was obtained from a corn to ethanol plant and used in a series of fermentations in four 11 L bioreactors. Treatments included three separate bacterial inoculants (A, B, and C) along with an untreated control. The inoculation treatments were fermented in the bioreactors for 48 hours while the untreated control was processed without additional storage or agitation that would have mimicked residence time in a bioreactor. All treatments were replicated four times and fresh, whole stillage was obtained each morning of the fermentations. Upon arrival at the lab, whole stillage was pH adjusted to 6.5 using 50% KOH and then cooled to 35° C. using an immersion chiller. Bioreactors were charged with 7,600 grams of pH adjusted, whole stillage and subsequently inoculated with one of the four bacterial treatments. Inoculum was prepared freshly before use by adding 2 grams of dried inoculum containing 5×10⁹ cfu per gram to 398 grams of di-ionized water and stirring well until suspended evenly. The bioreactors were then inoculated with the 400 g bacterial suspension to equal approximately 1.25×10⁶ cfu per gram of whole stillage. The bioreactors were agitated continuously while also temperature (35° C.) and pH controlled (pH 6.5 using 50% KOH) during the duration of a 48 hour fermentation period. Treatments A, B, and C consisted of Lactobacillus casei, Pediococcus acidophilus, and Lactobacillus plantarum, respectively. Bacteria inoculants were all obtained from AB Technologies located in Springfield, Oreg.

At the conclusion of the fermentation period, the whole stillage ferment was processed as follows. A sample of each whole stillage ferment was frozen for later laboratory analyses. The whole stillage ferment was centrifuged in a swing basket centrifuge at 1,000×g for 5 minutes using 1,000 ml polypropylene bottles to separate heavy solids from solubles, oils and waxes. This low speed and centrifuging time were intended to simulate separation results that would be found in a commercial plant utilizing a decanter. After centrifuging, supernatants of each bottle within treatments were combined into an 11 L plastic bucket with a spigot fastened to bottom of the sidewall. The heavy solids remaining in each bottle were carefully removed, combined within each treatment and weighed. From each batch of heavy solids (wet cake) within treatments, three subsamples of approximately 150 g each was taken for dry matter determination (50° C. 48 hours) and sample retention. The oils and waxes were allowed to separate from the solubles in the 11 L bucket for at least 2 hours and no more than 5 hours before the solubles fraction was bottom decanted from each bucket using the spigot. The decanting was stopped just before the oil and wax layer reached the bottom. Retained was mostly oils and waxes with a small portion of the remaining solubles. This remainder was transfer to a 500 ml separatory funnel and the oils or waxes adhering to the sidewall were rinsed into the funnel with diethyl ether. After the oil and waxes separated from the solubles in the separatory funnel, the solubles were bottom decanted more precisely and added to the previously separated solubles and a total weight was measured. The remaining oils and waxes were collected into 50 ml centrifuge tubes, all adhering to the sidewalls was rinsed out using diethyl ether. The collected oils and gums were then centrifuged lightly (1,000×g for 5 minutes) and the clear oil was decanted from the top into an additional tube. Both the waxes and the oils were then dried to evaporate ether before being weighed to record the amount of recoverable oil and waxes within treatment. Samples of the solubles were frozen for later lab analyses.

Samples of dried wet cake, frozen whole stillage and frozen solubles were sent to an outside laboratory (Dairyland Labs, Arcadia, Wis.) for chemical analyses. Ethanol soluble sugars were determined using 80% ethanol solution for extraction followed by a reaction with phenol-sulfuric acid and absorbance reading at 480 nanometers. Organic acids were determined using HPLC. Acid detergent and neutral fiber were determined by the VanSoest method.

Results are shown in Table 1. Treatment with each microorganism produced ethanol in significant quantities. Disappearance or digestion of nutrients resulted in production of end products including ethanol. Digestion of sugars, including dextrins and larger polysaccharides, was equal to about 58%. Surprisingly, about 20% of the neutral detergent fiber (“NDF”) was digestion during fermentation while no acid detergent fiber (“ADF”) disappearance was measured. The fraction NDF consists of cellulose, lignin, and hemicellulose, and ADF consists of cellulose and lignin. These data indicate the microbes utilized a large portion of hemicellulose to form end-products such as ethanol.

TABLE 1
CHEMICAL COMPOSITION OF WHOLE STILLAGE RE-FERMENTED FOR 48 HOURS
COMPARED WITH A CONTROL.

| | Treatment | | | | | | |
|---|---|---|---|---|---|---|---|
| | | | | | Trt | % | |
| | Control | Trt-A | Trt-B | Trt-C | Mean | Disappearance² | SEM¹ |
| ADF, % of dry matter | 3.84 | 4.79 | 4.21 | 4.66 | 4.55 | 0 | 1.21 |
| NDF, % of dry matter | 16.4 | 17.22 | 14.77 | 16.07 | 16.02 | 20 | |
| Ethanol soluble sugars, % of | 16.53^{q} | 6.80^{qs} | 6.20^{s} | 10.11^{qs} | 7.70 | 58 | 2.63 |
| dry matter | | | | | | | |
| Chlorine, % of dry matter | 0.68 | 0.87 | 0.77 | 0.62 | 0.75 | — | 0.15 |
| Ethanol, % of dry matter | 0.00^{a} | 10.95^{b} | 11.89^{b} | 12.48^{b} | 11.77 | | 0.75 |

^{abc}means within rows with unlike superscripts differ. P < 0.05.
^{qs}means within row with unlike superscripts differ. P < 0.10.
¹SEM—standard error of mean differences
²% Disappearance − (1 − % Cl in Control/% Cl in fermented sample) * 100, for each nutrient

### Example 2

A fermentation was conducted to produce additional ethanol from whole stillage byproduct. Whole stillage was obtained from a commercial corn to ethanol dry grind plant. Whole stillage is residual beer remaining after distillation and removal of ethanol from the beer after primary ethanol fermentation using corn mash. To a volume of about 5,000 gallons, an inoculum (L. plantarum) was added to equal approximately 1×10⁶ cfu's per gram of stillage after pH adjustment with calcium hydroxide (6.0). Anaerobsis was created by gassing the headspace with carbon dioxide and fermentation was commenced for a time of 56 hours. Samples were obtained approximately every 8 hours and analyzed for the items listed in Table 2. Residual carbohydrates were reduced over time and ethanol accumulated. Mass balance data (Table 3) indicated 40% of the fiber (cellulose and hemicellulose) along with all DP1, DP2, DP3 which refer to single, double and triple unit sugar polymers, respectively, and glycerol was converted to ethanol and presumably carbon dioxide. For example, DP1, DP2, and DP3 are representative of glucose, maltose, and dextrin, respectively.

TABLE 2
WHOLE STILLAGE FERMENTATION

| Fermentation | | | | | Lactic | | Acetic | Ethanol | |
|---|---|---|---|---|---|---|---|---|---|
| Time (hr.) | DP4+ | DP3 | DP2 | Glucose | Acid | Glycerol | Acid | (ppm) | pH |
| 0 | 1.0 | 0.1 | 0.4 | 0.0 | 0.3 | 1.3 | 0.1 | 48 | 5.7 |
| 8 | 0.9 | 0.1 | 0.5 | 0.0 | 0.3 | 1.3 | 0.1 | 50 | 5.6 |
| 16 | 0.9 | 0.1 | 0.0 | 0.0 | 1.0 | 1.3 | 0.1 | 100 | 6.0 |
| 24 | 0.9 | 0.0 | 0.0 | 0.0 | 1.1 | 1.3 | 0.1 | 129 | 5.7 |
| 32 | 0.7 | 0.0 | 0.0 | 0.0 | 1.4 | 1.3 | 0.1 | 1446 | 5.8 |
| 40 | 0.7 | 0.0 | 0.0 | 0.0 | 1.3 | 1.0 | 0.1 | 4957 | 5.8 |
| 48 | 0.7 | 0.0 | 0.0 | 0.0 | 1.2 | 0.3 | 0.1 | 9658 | 5.6 |
| 56 | 0.7 | 0.0 | 0.0 | 0.0 | 1.1 | 0.1 | 0.1 | 12153 | 5.5 |
| 59 (drop) | 0.7 | 0.0 | 0.0 | 0.0 | 1.1 | 0.0 | 0.1 | 12557 | 5.7 |

TABLE 3
RESULTS OF MASS BALANCE CALCULATIONS,
DISAPPEARANCE OF NUTRIENTS

| | | |
|---|---|---|
| Whole Stillage | % of Total | Biologically |
| Components | Solids | converted, % |

| | | | |
|---|---|---|---|
| Sugars | DP1 | 0.83 | 100 |
| | DP2 | 5.26 | 100 |
| | DP3 | 0.73 | 100 |
| | DP4 | 10.77 | 35 |
| | Glycerol | 9.2 | 100 |
| | Residual Starch | 3.5 | 88 |
| Fiber | Hemicellulose | 19.2 | 42 |
| | Cellulose | 10.7 | 42 |
| | Proteins | 27.0 | 39 |
| | Ash | 5.25 | |

Accordingly, the method described herein uses whole stillage from corn ethanol production and converts same to ethanol without any additional nutrient inputs. The method employs a fermentation with a bacterial microbe from the Order Lactobacillales. The process is anaerobic and fermentation with the microbe and selected byproducts results in ethanol, lactic acid and improved distillers grains feedstuffs.

Advantageously, ethanol can be economically produced using non-food materials using the method described herein. Various additional advantages are also gained by the present system. It was surprisingly found that in the method described herein the organism used converts multiple carbohydrate types, such as are found in the byproducts described herein, into ethanol, and does so during the same fermentation batch and in particular simultaneously converts the multiple carbohydrates to ethanol. Residual starches, fibers including cellulose and hemicelluloses, may all be converted to ethanol according to the method described herein with various extents of conversion. It was also surprisingly found that in the method described herein the microorganism converts multiple carbohydrate types into ethanol without the addition of enzymes. That is, the microbe disclosed herein advantageously converts multiple carbohydrates from multiple feedstocks simultaneously to ethanol and does so without added nutrients. An additional advantage is the application of a microbe from the Order Lactobacillales according to the method described herein is capable of producing industrial quantities of ethanol. It was also surprisingly found that in the method, glycerol is completely metabolized by the microbe and may be converted to ethanol.

It was surprisingly found that the resultant byproduct from the current method is higher in protein and digestible protein compared with the initial byproduct feedstock, particularly when the original feedstock is corn ethanol whole stillage. In addition, the byproduct from the current process is higher in rumen undegradable protein, thereby delivering a higher amount of metabolizable protein to the animal when fed to ruminants.

Advantageously, production of ethanol using the current method does not require sterilization of the byproduct feedstocks because the microbe used is robust and will out compete contaminating microbes. Thus, strict sterilization is not required. As a result, the production of ethanol according to the present method results in a significant cost savings.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It is also important to note that the construction and arrangement of the system, methods, and devices as shown or described in the various examples of embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements or materials, values of parameters, use of materials, colors, orientations, etc.) without materially departing from the novel teachings and advantages of the subject matter recited. For example, the order or sequence of any process or method steps may be varied or re-sequenced according to alternative embodiments. Other substitutions, modifications, changes and omissions may be made in the design, operating conditions and arrangement of the various examples of embodiments without departing from the spirit or scope of the present inventions.

While this invention has been described in conjunction with the examples of embodiments outlined above, various alternatives, modifications, variations, improvements and/or substantial equivalents, whether known or that are or may be presently foreseen, may become apparent to those having at least ordinary skill in the art. Accordingly, the examples of embodiments of the invention, as set forth above, are intended to be illustrative, not limiting. Various changes may be made without departing from the spirit or scope of the invention. Therefore, the invention is intended to embrace all known or earlier developed alternatives, modifications, variations, improvements and/or substantial equivalents.

The technical effects and technical problems in the specification are exemplary and are not limiting. It should be noted that the embodiments described in the specification may have other technical effects and can solve other technical problems.

1. A method of producing ethanol, the method comprising fermentation of carbohydrates from multiple byproduct feedstocks simultaneously during the same fermentation batch by application of a bacterial microbe which converts said carbohydrates to ethanol.

2. The method of producing ethanol of claim 1, wherein the byproduct feedstocks are obtained from whole stillage.

3. The method of producing ethanol of claim 1, wherein the bacterial microbe is from the Order Lactobacillales.

4. The method of producing ethanol of claim 3, wherein the bacterial microbe is selected from the group consisting of Lactobacillus casei, Pediococcus acidophilus, and Lactobacillus plantarum.

5. The method of producing ethanol of claim 1, wherein the bacterial microbe converts multiple carbohydrate types into ethanol without the addition of enzymes.

6. The method of producing ethanol of claim 1, wherein the bacterial microbe converts multiple carbohydrate types into ethanol without pre-treatment.

7. A method of producing ethanol from whole stillage comprising converting multiple carbohydrates from multiple feedstocks simultaneously into ethanol without pre-treatment and without added enzymes.

8. The method of producing ethanol of claim 7, wherein the method includes the addition of a bacterial microbe.

9. The method of producing ethanol of claim 8, wherein the bacterial microbe is from the Order Lactobacillales.

10. The method of producing ethanol of claim 9, wherein the bacterial microbe is selected from the group consisting of Lactobacillus casei, Pediococcus acidophilus, and Lactobacillus plantarum.

11. A method of producing ethanol comprising application of a microbe from the Order Lactobacillales to a byproduct so as to produce ethanol.

12. The method of claim 11, wherein the method produces industrial quantities of ethanol.

13. The method of producing ethanol of claim 11, wherein the byproduct is a feedstock obtained from whole stillage.

14. The method of producing ethanol of claim 11, wherein the microbe is selected from the group consisting of Lactobacillus casei, Pediococcus acidophilus, and Lactobacillus plantarum.

15. The method of producing ethanol of claim 11, wherein the microbe converts multiple carbohydrate types into ethanol without the addition of enzymes.

16. The method of producing ethanol of claim 11, wherein the microbe converts multiple carbohydrate types into ethanol without pre-treatment.
